(19) United States
(12) Patent Application Publication
(10) Pub. No.: US 2022/0370615 A1
(43) Pub. Date: Nov. 24, 2022

(54) Prevention of Illicit Manufacture of Methamphetamine from Pseudoephedrine Using Food Flavor Excipients

(71) Applicant: Raghavan Rajagopalan, St. Peters, MO (US)

(72) Inventor: Raghavan Rajagopalan, St. Peters, MO (US)

(73) Assignee: Satara Pharmaceuticals, St Peters, MO (US)

(21) Appl. No.: 17/306,943

(22) Filed: May 4, 2021

(51) Int. Cl. A61K 47/22; A61K 31/137

(52) U.S. Cl. CPC A61K 47/22; A61K 31/137

(57) ABSTRACT

The invention relates generally to ephedrine or pseudoephedrine compositions containing biocompatible organoleptic (food flavoring) excipients that would prevent the illicit manufacture of methamphetamine from ephedrine or pseudoephedrine.

This is a divisional application of the previously filed non-provisional application Ser. No. 14/499,141 filed on Sep. 28, 2014, and said application is incorporated herein by reference. This divisional application claims benefit of priority based on the non-provisional application Ser. No. 14/499,141, which claims benefit of priority based on provisional application No. 61/884,304 filed on Sep. 30, 2013.

### CROSS-REFERENCE TO RELATED APPLICATIONS

Non-provisional application Ser. No. 14/499,141.

### STATEMENT REGARDING FEDERALLY SPONSORED RESEARCH OR DEVELOPMENT

Not applicable.

### THE NAMES OF THE PARTIES TO A JOINT RESEARCH AGREEMENT

Not applicable.

### INCORPORATION-BY-REFERENCE OF MATERIAL SUBMITTED ON A COMPACT DISC

Not Applicable.

### FIELD OF THE INVENTION

This invention relates to compositions and the method of preventing illicit manufacture of methamphetamine. Particularly, this invention relates to pseudoephedrine or ephedrine compositions containing food flavor excipients that chemically suppress the conversion of pseudoephedrine or ephedrine to methamphetamine.

### PRELIMINARY NOTE

Various prior art references in the specification are indicated by italicized Arabic numerals in brackets. Full citation corresponding to each reference number is listed at the end of the specification, and is herein incorporated by reference in its entirety in order to describe fully and clearly the state of the art to which this invention pertains. Unless otherwise specified, all technical terms and phrases used herein have their art-recognized meaning, which can be found by reference to standard texts, journal references, and contexts known to those skilled in the art as established by International Union of Pure and Applied Chemistry (IUPAC), the American Chemical Society (ACS), and other international professional societies. The rules of nomenclature are described in various publications, including, “Nomenclature of Organic Compounds,” [1], and “Systematic Nomenclature of Organic Chemistry” [2], which are herein incorporated by reference in their entireties.

### BACKGROUND

Pseudoephedrine (1) is a highly effective medication for temporary relief of nasal decongestion due to cold and seasonal allergies. It is the active pharmaceutical ingredient (API′ or ‘drug substance’) in the over-the-counter (OTC) allergy medications such as Sudafed,™ Sinufed,™ and Novafed.™ However, due to its widespread abuse in the illicit manufacture of methamphetamine (2), the distribution of this medication has been severely

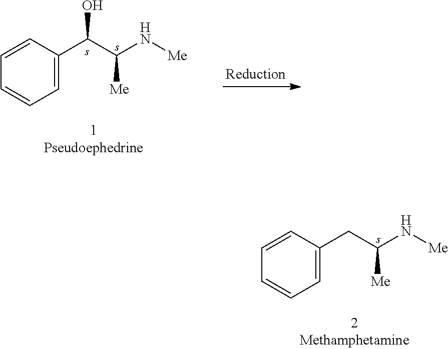

restricted and controlled, thereby placing undue burden on the individuals suffering from nasal congestion and other related discomforts from being able to readily obtain this highly effective medication OTC. Due to high addiction potential of methamphetamine, clandestine laboratories for its illicit manufacture have proliferated worldwide thereby imposing an enormous socioeconomic burden to the society. Unfortunately, such illicit manufacture is also dangerous which results in serious bodily injury or death to the victims, and presents formidable safety problems for law enforcement officials. Therefore, development of an effective abuse-deterrent composition comprising pseudoephedrine and an additive (referred to as ‘excipient) that would substantially suppress or block the chemical conversion of pseudoephedrine or its diastereomer, ephedrine, to methamphetamine remains an urgent unmet need.

Recently, various solid dosage formulations such as Tarex™ and Nexafed™ (referred to as ‘tamper-resistant’ technology) which physically impedes the extraction of pseudoephedrine from such formulations have been recently introduced by Highland Pharmaceuticals and Acura Pharmaceuticals respectively. However, these physical methods have serious limitation in that they cannot prevent the chemical transformation of pseudoephedrine to methamphetamine. Accordingly, the object of the present invention is to develop a safe, biocompatible formulation that would chemically prevent conversion of pseudoephedrine to methamphetamine under the attempted reaction conditions regardless of the amount of the API is extracted from the formulation.

Illicit manufacture of methamphetamine from pseudoephedrine and ephedrine has been accomplished by numerous methods as described in a recent book entitled, “Secrets of Methamphetamine Manufacture” [3] that involves two-electron reduction of pseudoephedrine or ephedrine with various reducing agents including lithium, zinc, and phosphorous. as illustrated in FIG. 1 [4, 5]. The most popular method is the ‘soda-bottle shake and bake’ procedure using lithium and ammonium nitrate [6]. The reagents and the common solvents such as ether, toluene, light petroleum, ammonia, hydrochloric acid, hydriodic acid, sodium hydroxide, and the like are readily accessible to the illicit manufacturers. For example, red phosphorus and lithium metal are obtained lithium batteries match boxes respectively. The precise mechanism of reduction of ephedrine or pseudoephedrine is complex and has not yet been fully elucidated, but can be rationalized by a process involving several reactive intermediates, 3, 4, 5, 7, 8, and 9 as shown in FIG. 1. Therefore, any entity that would either combine with or prevent the formation of said reactive species would obviate or effectively inhibit the formation of methamphetamine. Although there are enormous number of molecules containing unsaturated functionalities (e.g. double and triple bonds, carbonyl groups, and the like) that could, in principle, be employed for the intended purpose, the most desirable are the ones that are generally recognized as safe (‘GRAS’) by the United States Food and Drug Administration (FDA). Accordingly, regardless of the source of the starting materials or of the competency of the illicit manufacturer, this invention relates to pseudoephedrine or ephedrine formulations comprising food flavoring excipients (referred to as ‘organoleptic’ agents or ‘organoleptics’) that are biocompatible and non-toxic.

### SUMMARY

The present invention relates to pharmaceutically acceptable compositions of for preventing or inhibiting the formation of methamphetamine comprising:

(a) ephedrine or pseudoephedrine and;

(b) at least one organoleptic excipient;

wherein the amount of said excipient is sufficient to inhibit effectively the formation of methamphetamine. The term ‘effectively’ herein implies that the amount of methamphetamine, if formed, should be less than about 25%.

One embodiment of the present invention relates to pharmaceutically acceptable compositions comprising ephedrine or pseudoephedrine and one or more pyrazine-based excipient of Formula I,

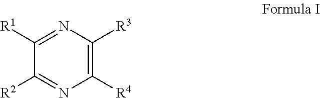

wherein R¹ to R⁴ are selected from the group consisting of hydrogen, C1-C10 alkyl, C1-C10 acyl, C1-C10 hydroxyalkyl, C1-C10 alkoxy, C1-C10 alkoxyalkyl, C1-C10 alkylthio, and C1-C10 alkoxycarbonyl.

Another embodiment of the present invention relates to pharmaceutically acceptable compositions comprising ephedrine or pseudoephedrine and one or more biocompatible pyrazine-based excipient of Formula II, wherein R¹ and R² are independently selected from the group consisting of hydrogen, C1-C10 alkyl, C1-C10 acyl, C1-C10 hydroxyalkyl, C1-C10 alkoxy, C1-C10 alkoxyalkyl, C1-C10 alkylthio, and C1-C10 alkoxycarbonyl. X and Y are independently

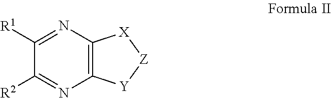

—CHR⁵—, —O—, —N—, —NR⁶—, or —S—; Z is —(CH₂)ₘ—, —C(R⁸)═, or ═CR⁹—CR¹⁰═; subscript ‘m’ varies from 0 to 4. R⁵ to R¹⁰ are independently selected from the group consisting of hydrogen, C1-C10 alkyl, C1-C10 acyl, C1-C10 hydroxyalkyl, C1-C10 alkoxy, and C1-C10 alkoxycarbonyl.

### BRIEF DESCRIPTION OF THE DRAWINGS

FIG. 1. Mechanistic scheme for the reduction of pseudoephedrine.

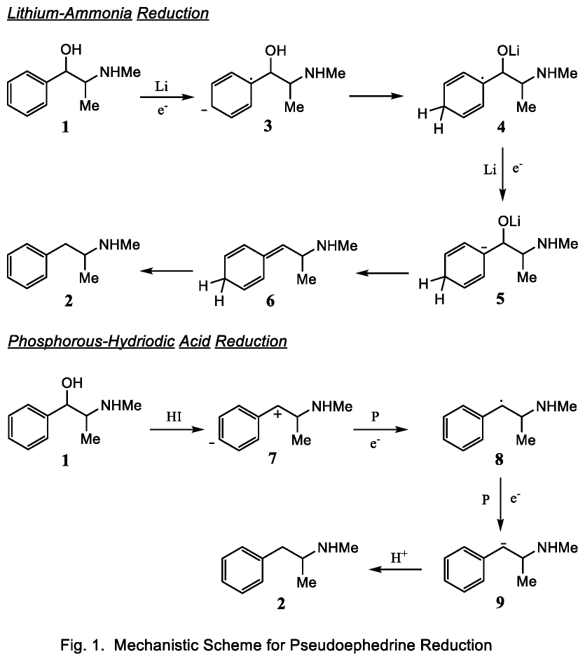

### DETAILED DESCRIPTION

The invention discloses a chemical method to prevent the conversion of ephedrine or pseudoephedrine to methamphetamine. Our proposition is based on introducing a suitable natural or artificial food flavoring exipient into the formulation such that said excipient may capture the electrons from the reducing agents (e.g. lithium or phosphorus) at a much higher rate than pseudoephedrine or may combine with the reactive species 3, 4, 5, or 7 generated from the reduction of pseudoephedrine thereby blocking the formation of methamphetamine under the reaction conditions employed by the illicit manufacturer. To the best of our knowledge, such an approach has not been disclosed before. It is important to note that although fruit-flavored pseudoephedrine formulations such as raspberry- or grape-flavored are well known [7], the amount of flavoring agent in these compositions are typically small compared to the amount of API, and hence, would not be very effective in blocking the conversion of pseudoephedrine to methamphetamine. Based on the stoichiometry, it would require at least an equivalent mole of the excipient to be able to block the formation of methamphetamine completely. Various flavoring agents such as pyrimidines, furans, oxazolines, thiophenes, thiazolidines, thiazoles, and the like, and their organoleptic properties are described in detail in “Food Flavoring Processes” [8], which is incorporated by reference in its entirety. Among the numerous organoleptic compounds that are known and used [8, 9], pyrazine derivatives present an attractive choice for the intended purpose because they are: (a) widely distributed in edible plants (e.g. potato, bell pepper, corn, peanut, etc.); (b) have been used extensively as flavor enhancing agents in variety of foods (ice cream, milk pudding, popcorn, beef and chicken broth, etc.); (c) belong to the category of substances that are generally recognized as safe (GRAS) by the USFDA [10]; and (d) are very effective in blocking the formation of methamphetamine from pseudoephedrine, as will be demonstrated later in the Examples section.

The present invention relates to pharmaceutically acceptable compositions of for preventing or inhibiting the formation of methamphetamine comprising:
(c) ephedrine or pseudoephedrine and;
(d) at least one organoleptic excipient;
   wherein the amount of said excipient is sufficient to inhibit effectively the formation of methamphetamine. The term ‘effectively’ herein implies that the amount of methamphetamine, if formed, should be less than about 25%.

One embodiment of the present invention relates to pharmaceutically acceptable compositions comprising:
(a) the API's ephedrine or pseudoephedrine and;
(b) at least one biocompatible organoleptic exipient selected from the group consisting of pyrazines, pyrimidines, furans, oxazolines, thiophenes, thiazolidines, and thiazoles.

Another embodiment of the present invention relates to the excipients of Formula I, wherein R¹ to R⁴ are hydrogen, C1-C10 alkyl, C1-C10 acyl, hydroxyl, C1-C10 hydroxyalkyl, C1-C10 alkoxy, C1-C10 alkoxyalkyl, C1-C10 alkylthio, carboxyl, and C1-C10 alkoxycarbonyl.

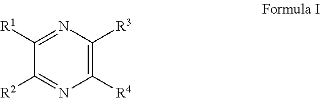

Another embodiment relates to the excipients of Formula I, wherein R¹ to R⁴ are independently hydrogen, C1-C10 alkyl, C1-C10 acyl, or C1-C10 alkoxyl.

Another embodiment relates to the excipients of Formula I, wherein R¹ is C₁-C10 acyl; and R² to R⁴ are independently hydrogen, or C₁-C10 alkyl.

Another embodiment relates to the excipients of Formula I, wherein R¹ is C₁-C10 alkoxy; and R² to R⁴ are independently hydrogen, or C₁-C10 alkyl.

Another embodiment relates to the excipients of Formula I, wherein R¹ is acetyl; and R² to R⁴ are hydrogen or methyl.

Another embodiment relates to the excipients of Formula I, wherein R¹ is methoxy or ethoxy; and R² to R⁴ are hydrogen or methyl.

Another embodiment relates to the excipients of Formula II, wherein R¹ and R² are

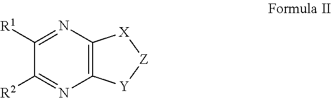

independently selected from the group consisting of hydrogen, C1-C10 alkyl, C1-C10 acyl, C1-C10 hydroxyalkyl, C1-C10 alkoxy, C1-C10 alkoxyalkyl, C1-C10 alkylthio, and C1-C10 alkoxycarbonyl. X and Y are independently selected from the group consisting of —(CH₂)ₘ—, —(CHR⁵)—, —O—, —N—, —NR⁶—, or —S—; Z is —(CHR⁷)—, —C(R⁸)═, or ═C(R⁹)—C(R¹⁰)═; subscript ‘m’ varies from 0 to 4. R⁵ is selected from the group consisting of C1-C10 alkyl, C1-C10 acyl, C1-C10 hydroxyalkyl, and C1-C10 alkoxycarbonyl. R⁶ is selected from the group consisting of hydrogen, C1-C10 alkyl, C1-C10 acyl, C1-C10 hydroxyalkyl, and C1-C10 alkoxycarbonyl. R⁷ to R¹⁰ are independently selected from the group consisting of hydrogen, C1-C10 alkyl, C1-C10 acyl, C1-C10 hydroxyalkyl, C1-C10 alkoxy, and C1-C10 alkoxycarbonyl.

Another embodiment relates to the excipients of Formula II, wherein R¹ and R² are independently hydrogen, C1-C10 alkyl, C1-C10 acyl, or C1-C10 alkoxy; R⁵ and R⁶ are independently hydrogen or C1-C10 alkyl; X is —(CH₂)ₘ—, Y is —(CH₂)ₘ—, —O—, —NR⁵—, or —S—; Z is —(CHR⁵)—, —C(R⁵)═, or ═C(R⁵)—C(R⁶)═; and ‘m’ varies from 0 to 2.

Another embodiment relates to the excipients of Formula II, wherein R¹ and R² are independently hydrogen, C1-C10 alkyl, C1-C10 acyl, or C1-C10 alkoxy; R⁵ and R⁶ are independently hydrogen or C1-C10 alkyl; X is —(CHR⁵)—, Y is —(CH₂)ₘ—, —O—, —NR⁵—, or —S—; Z is —C(R⁵)═, and ‘m’ varies from 0 to 2.

Another embodiment relates to the excipients of Formula II, wherein R¹ and R² are independently hydrogen, C1-C10 alkyl, C1-C10 acyl, or C1-C10 alkoxy; R⁵ and R⁶ are independently hydrogen or C1-C10 alkyl; X and Y are —(CHR⁵)—; Z is ═C(R⁵)—C(R⁶)═; and ‘m’ varies from 0 to 2.

Another embodiment relates to the excipients of Formula II, wherein R¹ and R² are independently hydrogen, C1-C10 alkyl, C1-C10 acyl, or C1-C10 alkoxy; R⁵ and R⁶ are independently hydrogen or C1-C10 alkyl; X and Y are —N—, Z is ═C(R⁵)—C(R⁶)═; and ‘m’ varies from 0 to 2.

Another embodiment relates to the excipients of Formula II, wherein R¹ and R² are independently hydrogen, C1-C10 alkyl, C1-C10 acyl, or C1-C10 alkoxy; R⁵ and R⁶ are independently hydrogen or C1-C10 alkyl; X is —(CHR⁵)—; Y is —N—, Z is ═C(R⁵)—C(R⁶)═; and ‘m’ varies from 0 to 2.

Another embodiment relates to the excipients of Formula II, wherein R¹ and R² are independently hydrogen, C1-C10 alkyl, C1-C10 acyl, or C1-C10 alkoxy; R⁵ and R⁶ are independently hydrogen or C1-C10 alkyl; X and Y are —(CHR⁵)—; —N—, Z is ═C(R⁵)—C(R⁶)═; and ‘m’ varies from 0 to 2.

As used herein, the term “pharmaceutically acceptable” means approved by a regulatory agency of an appropriate federal or state government; or listed in the U.S. Pharmacopoeia or other generally recognized pharmacopoeia for use in animals, and more particularly in humans; or does not impart significant deleterious or undesirable effect on a subject to whom it is administered and in the context in which it is administered.

Compositions of the present invention can be formulated in the form of solutions, suspensions, emulsions, tablets, pills, capsules, powders, controlled- or sustained-release formulations and the like [13]. Such formulations will contain a therapeutically effective amount of the active pharmaceutical ingredient (API) comprising ephedrine derivatives including ephedrine, pseudoephedrine, norephedrine, and norpseudoephedrine together with a suitable amount of organoleptic exipient of Formulas I and II so as to provide the form for proper administration to the patient. The composition of the present invention is preferably administered orally in the form of tablets, capsules, solutions, or suspensions. Optionally, compositions of the invention further comprise one or more pharmaceutically acceptable electrolytes, salts, carriers, binders, coatings, preservatives and/or excipients auxiliaries, adjuvants diluents, surfactants, buffers, electrolytes, salts, carriers, binders, coatings, or preservatives as would be understood in the art. Preferably, the components meet the standards of the National Formulary (“NF”), United States Pharmacopoeia (“USP”; United States Pharmacopeia Convention Inc., Rockville, Md.), or Handbook of Pharmaceutical Manufacturing Formulations.

Solid dosage forms for oral administration include, for example, capsules, tablets, gelcaps, pills, dragees, troches, powders, granules, and lozenges. In such solid dosage forms, the compounds or pharmaceutically acceptable salts thereof can be combined with one or more pharmaceutically acceptable carriers. The compounds and pharmaceutically acceptable salts thereof can be mixed with carriers including, but not limited to, lactose, sucrose, starch powder, corn starch, potato starch, magnesium carbonate, microcrystalline cellulose, cellulose esters of alkanoic acids, cellulose alkyl esters, talc, stearic acid, magnesium stearate, magnesium oxide, sodium and calcium salts of phosphoric and sulfuric acids, sodium carbonate, agar, mannitol, sorbitol, sodium saccharin, gelatin, acacia gum, alginic acid, sodium alginate, tragacanth, colloidal silicon dioxide, croscarmellose sodium, polyvinylpyrrolidone, and/or polyvinyl alcohol, and then tableted or encapsulated for convenient administration. Such capsules or tablets can contain a controlled-release formulation, as can be provided in a dispersion of the compound or salt in hydroxypropylmethyl cellulose. In the case of capsules, tablets, and pills, the dosage forms also can include buffering agents, such as sodium citrate, or magnesium or calcium carbonate or bicarbonate. Tablets and pills additionally can, for example, include a coating (e.g., an enteric coating) to delay disintegration and absorption.

Liquid dosage forms of the compounds of the invention for oral administration include, for example, pharmaceutically acceptable emulsions, solutions, suspensions, syrups, and elixirs containing inert diluents commonly used in the art (e.g., water). Such compositions also can include adjuvants, such as wetting, emulsifying, suspending, flavoring (e.g., sweetening), and/or perfuming agents.

Additional components can be included in the compositions of this invention for various purposes generally known in the pharmaceutical industry. These components tend to impart properties that, for example, enhance retention of the active pharmaceutical ingredient or salt at the site of administration, protect the stability of the composition, control the pH, facilitate processing of the API, and the like. Specific examples of such components include surface; active, wetting, or emulsifying agents (e.g., lecithin, polysorbate-80, TWEEN 80, pluronic 60, and polyoxyethylene stearate); preservatives (e.g., ethyl-p-hydroxybenzoate); microbial preservatives (e.g., benzyl alcohol, phenol, m-cresol, chlorobutanol, sorbic acid, thimerosal, and paraben); agents for adjusting pH or buffering agents (e.g., acids, bases, sodium acetate, sorbitan monolaurate, etc.); agents for adjusting osmolarity (e.g., glycerin); thickeners (e.g., aluminum monostearate, stearic acid, cetyl alcohol, stearyl alcohol, guar gum, methyl cellulose, hydroxypropylcellulose, tristearin, cetyl wax esters, polyethylene glycol, etc.); colorants; dyes; flow aids; non-volatile silicones (e.g., cyclomethicone); clays (e.g., bentonites); adhesives; bulking agents; flavorings; sweeteners; adsorbents; fillers (e.g., sugars such as lactose, sucrose, mannitol, sorbitol, cellulose, calcium phosphate, etc.); diluents (e.g., water, saline, electrolyte solutions, etc.); binders (e.g., gelatin; gum tragacanth; methyl cellulose; hydroxypropyl methylcellulose; sodium carboxymethyl cellulose; polyvinylpyrrolidone; sugars; polymers; acacia; starches, such as maize starch, wheat starch, rice starch, and potato starch; etc.); disintegrating agents (e.g., starches, such as maize starch, wheat starch, rice starch, potato starch, and carboxymethyl starch; cross-linked polyvinyl pyrrolidone; agar; alginic acid or a salt thereof, such as sodium alginate; croscarmellose sodium; crospovidone; etc); lubricants (e.g., silica; talc; stearic acid and salts thereof, such as magnesium stearate; polyethylene glycol; etc.); coating agents (e.g., concentrated sugar solutions including gum arabic, talc, polyvinyl pyrrolidone, carbopol gel, polyethylene glycol, titanium dioxide, etc.); and antioxidants (e.g., sodium metabisulfite, sodium bisulfite, sodium sulfite, dextrose, phenols, thiophenols, etc.).

Binding agents include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose, calcium carbonate, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof. Suitable forms of microcrystalline cellulose include, for example, AVICEL-PH-101, AVICEL-PH-103 and AVICEL-PH-105.

Fillers include, but are not limited to, talc, calcium carbonate (e.g., granules or powder), lactose, microcrystalline cellulose, powdered cellulose, dextrates, kaolin, mannitol, silicic acid, sorbitol, starch, pre-gelatinized starch, and mixtures thereof.

Liquid preparations for oral administration can take the form of solutions, syrups or suspensions. Alternatively, the liquid preparations can be presented as a dry product for constitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and/or preservatives (e.g., methyl or propyl-p-hydroxybenzoates or sorbic acid). The preparations can also contain buffer salts, flavoring, coloring, perfuming and sweetening agents as appropriate. In addition, a composition of the present invention can be formulated for buccal administration in the form of tablets or lozenges formulated in a conventional manner. Other methods of oral delivery of the composition of the invention will be known to the skilled artisan and are within the scope of the invention.

The invention is further detailed in the following Examples, which are offered by way of illustration and are not intended to limit the scope of the invention in any manner. These examples demonstrate that the excipients of Formulas I and II are effective in eliminating or substantially diminishing the formation of methamphetamine from pseudoephedrine and ephedrine.

### Example 1

General Procedure for the Reaction of the Exipient of Formulas I and II and Pseudoephedrine with Lithium in Liquid Ammonia.

Lithium metal pieces (35 mg, 5.0 mmol) were carefully added to liquid ammonia (5-10 mL) at about −78° C. (dry-ice, isopropyl alcohol). A dark blue solution began to form within a few seconds. The dark blue solution was stirred at −78° C. for about 3 to 5 minutes. Thereafter, a mixture of the exipient of Formula I (122-166 mg, 1.0 mmol) and pseudoephedrine (165 mg, 1.0 mmol) in anhydrous tetrahydrofuran (3 mL) was added to the dark blue solution. The entire reaction mixture was stirred while the temperature of the reaction was slowly raised to about −20° C. Stirring was continued at this temperature for about 1 hour. The reaction mixture was quenched with saturated ammonium chloride (2 mL) and water (3 mL), and diluted with methanol (5 mL). The reaction mixture was analyzed and quantified for the presence of methamphetamine. The results are given in Example 3 below.

### Example 2

General Procedure for the Reaction of the Exipient of Formulas I and II and Pseudoephedrine with Red Phosphorous and Hydriodic Acid.

A mixture of the exipient of Formula I (122-166 mg, 1.0 mmol) and pseudoephedrine (165 mg, 1.0 mmol) in 57% aqueous hydriodic acid (2 mL) was treated with red phosphorous (50 mg, 1.6 mmol), and the mixture was stirred and heated at 90-100° C. for 1-1.5 hours. The reaction mixture was cooled to ambient temperature and diluted with water (2 mL) and methanol (5 mL). The reaction mixture was analyzed and quantified for the presence of methamphetamine. The results are given in Example 3 below.

### Example 3

The effect of various excipients in the conversion of pseudoephedrine to methamphetamine is given in Table 1. Clearly, the pyrazine-based excipients are effective in inhibiting the conversion of pseudoephedrine to methamphetamine, with acetypyrazine being the most effective among all of the pyrazine derivatives tested. It should be all of the excipients in Table 1 are safe [8], and have been used extensively as food flavors in ice cream, pudding, bread, etc. In, particular, the LD₅₀ of acetylpyrazine is >4000 mg/kg. The present invention is not limited to the use of pyrazine-based organoleptics. Other classes of compounds that are used in food flavors described previously can be used can also be used effectively to suppress the formation of methamphetamine.

TABLE 1
Relative percentage of methamphetamine formation.

| | Percent Methamphetamine | |
|---|---|---|
| Excipient | Li-NH₃ Reduction | P-HI Reduction |
| None (Pseudoephedrine only) | 100 | 100 |
| Acetylpyrazine | <<1 | 6 |
| 2-Ethoxy-5-methylpyrazine | 7 | 15 |
| 2-Ethoxy-6-methylpyrazine | 4 | 13 |
| 2-Isobutyl-3-methoxypyrazine | 2 | 27 |
| 2-Methoxy-3-methylpyrazine | <1 | 2.0 |
| 2,3,5-Trimethylpyrazine | 8 | 20 |
| 2-Acetylthiazole | <1 | |
| Isobutyl anthranilate | <1 | 2.3 |

### Example 4

### Typical Procedure for the Preparation of Typical Bulk Solid Dosage Form for Abuse-Deterrent Pseudoephedrine Formulation.

(a) Preparation of Bulk Powder.

A mixture of pseudoephedrine hydrochloride (120.00 g), acetylpyrazine (180.00 g), starch (5.49 g), PVP (7.86 g), SOW (3.2 g), and, optionally p-acetamidophenol (‘actetaminophen’) (328.45 g) is treated with sufficient amount of water (c.a. 645-1500 mL) to yield a suspension comprising 30-50% of solid materials. This slurry is then pumped to a spray dryer targeting for a final moisture content of about 1.0%. Slurry feed rate, inlet temperature, air flow, and atomization air pressure of the spray dryer will be carefully controlled to produce a powder form of the drug product with particle size with optimum flow and compressibility properties. The resulting product is collected in the packaging cyclone, and is appropriately packaged and labeled for tabletting.

Although the above formulation contains acetaminophen, the present invention cannot be construed as limiting; other analgesics and antipyretics such as ibuprofen, naproxen, aspirin, and the like can be substituted for acetaminophen, or, optionally, the pseudoephedrine can be formulated without any analgesics or antipyretics. Furthermore, the exipient is also not limited to acetylpyrazine; other food flavoring agents derived from pyrimidines, furans, oxazolines, thiophenes, thiazolidines, and thiazoles can be used.

(b) Preparation of Tablets.

The resulting spray dried powder from Step (a) is typically put into a hopper, which will feed a high speed tablet press. Tooling and press set up parameters include: die fill depth, compression force, press speed, ejection forces. These parameters will affect tablet weight, dosage, thickness, hardness, tablet friability, disintegration time, and dissolution rate. Tooling size and dimension may vary to result in a tablet, which can be easily swallowed by the patient.

The typical composition of one abuse-deterrent pseudoephedrine tablet is given in Table 1 below.

TABLE 1
Ingredients in One Abuse-Deterrent Pseudoephedrine Tablet

| Ingredient | Amount (mg) | Amount (mg) |
|---|---|---|
| Pseudoephedrine HCL | 120 | 120 |
| Acetylpyrazine | 180 | 180 |
| APAP Code 7375 | None | 328 |
| Starch 1500 | 9 | 6 |
| PVP | 8 | 8 |
| SOW | 3 | 3 |
| Total Tablet Weight | 320 | 645 |

### Example 5

### Typical Procedure for the Preparation of Typical Bulk Liquid Dosage Form for Abuse-Deterrent Pseudoephedrine Formulation.

A mixture of pseudoephedrine hydrochloride (12.0 g), acetylpyrazine (18.0 g) and, optionally, p-acetamidophenol (‘actetaminophen’) (32.8 g) in glycerin (200.0 g) and propylene glycol (30.0 g) is stirred at ambient temperature until all the solids have dissolved. Thereafter, sorbitol (200.0 g), sucrose (100.0 g), and peppermint flavor (0.1 g) are added, and the entire mixture is diluted with sufficient water to a final volume of 2 liters.

The typical composition of a 10-mL pseudoephedrine solution/syrup is given in Table 2 below.

TABLE 2
Ingredients in a 10-mL Abuse-Deterrent Pseudoephedrine Syrup

| Ingredient | Amount (mg) | Amount (mg) |
|---|---|---|
| Pseudoephedrine HCL | 60 | 60 |
| Acetylpyrazine | 90 | 90 |
| APAP Code 7375 | None | 164 |
| Glycerine | 1000 | 1000 |
| Propylene glycol | 150 | 150 |
| Sorbitol (70% Solution) | 1000 | 1000 |
| Sucrose | 500 | 500 |
| Peppermint flavor | 5 | 5 |

Although the above formulation contains acetaminophen, the present invention cannot be construed as limiting; other analgesics and antipyretics such as ibuprofen, naproxen, aspirin, and the like can be substituted for acetaminophen, or, optionally, the pseudoephedrine can be formulated without any analgesics or antipyretics. The exipient is also not limited to acetylpyrazine; other food flavoring agents derived from pyrimidines, furans, oxazolines, thiophenes, thiazolidines, and thiazoles can be used. Finally, the pseudoephedrine syrup may be formulated without any sweeteners, or the sucrose may be substituted with artificial sweeteners such as saccharin, sucralose, aspartame, and the like.

### REFERENCES

1. Fox, R. B.; Powell, W. H. Nomenclature of Organic Compounds: Principles and Practice, Second Edition. Oxford University Press: Oxford, 2001.
2. Hellwinkel, D. Systematic Nomenclature of Organic Chemistry: A Directory of Comprehension and Application of its Basic Principles. Springer-Verlag, Berlin, 2001.
3. Fester, U. Secrets of Methamphetamine Manufacture. Festering Publications: Greenbay, Wis., 2009.
4. Skinner, H. F. Methamphetamine synthesis via hydriodic acid/red phosphorus reduction of ephedrine. Forensic Science International 1990, 48(2), 123-134.
5. Ely, R. A.; McGrath, D. C. Lithium-ammonia reduction of ephedrine to methamphetamine: an unusual clandestine synthesis. Journal of Forensic Sciences 1990, 35(3), 720-723.
6. Leech, R. L. Stabilization of one-pot methamphetamine synthesis systems. U.S. Patent Application 2014, 2014/0034885.
7. McCabe, T. T. et al. Flavored film-coated tablet. U.S. Patent 1992, U.S. Pat. No. 5,098,715.
8. Piatauro, N. D. Food Flavoring Processes. Noyes Data Corporation: Park Ridge, 1976.
9. Bellanca, N.; Furia, T. E. (Eds.). Feraroli's Handbook of Flavor Ingredients, Second Edition. CRC Press: Cleveland, Ohio, 1975.
10. Adams, T. B. et al. The FEMA GRAS assessment of pyrazine derivatives used as flavor ingredients. Food and Chemical Toxicology 2002, 40, 429-451.
11. Leech, R. L. et al. Methods and compositions for deterring abuse. U.S. Patent Application 2011, 2011/0077238.
12. Brzeczko, A. W. Pharmaceutical compositions for deterring misuse, abuse, and diversion. PCT 2011, WO2011079074.
13. Niazi, S. K. (Ed.). Handbook of Pharmaceutical Formulations. CRC Press: Cleveland, Ohio, 2004.

26. A method for deterring illicit manufacture of methamphetamine from ephedrine or pseudoephedrine, said method comprising:
(a) selecting an active pharmaceutical ingredient, wherein the active pharmaceutical ingredient is ephedrine or pseudoephedrine;
(b) selecting one or more inactive ingredients capable of forming solid or liquid dosage formulations with the active ingredient; wherein said inactive ingredients comprise pharmaceutically acceptable binders, carriers, buffers, salts, coatings, diluents, adjuvants, and preservatives;
(c) introducing one or more organoleptic excipient into a mixture comprising said active and inactive ingredients; wherein said organoleptic excipient is capable of chemically inhibiting the reduction of said active ingredients with reducing agents comprising alkali metals, zinc, and phosphorous; and wherein said organoleptic excipient is selected from the group consisting of pyrazines, pyrimidines, thiazolines, thiazolidines, and thiazoles;
(d) optionally adding a pharmaceutically acceptable analgesic or antipyretic agent to the mixture in step (c); and
(e) preparing pharmaceutically acceptable solid or liquid dosage forms comprising the active ingredient, the organoleptic excipient, and the inactive ingredient, and, optionally, the analgesic or the antipyretic agent.

27. The method of claim 26 wherein the organoleptic excipient is selected from the group consisting of pyrazines described by structural Formula I, 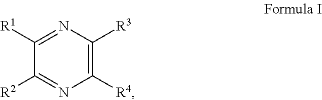
wherein each of R¹, R² R³, and R⁴ is independently selected from the group consisting of hydrogen, C1-C10 alkyl, C1-C10 acyl, C1-C10 hydroxyalkyl, C1-C10 alkoxy, C1-C10 alkoxyalkyl, C1-C10 alkylthio, and C1-C10 alkoxycarbonyl; and structural Formula II, 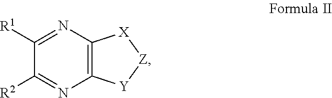
wherein each of R¹ and R² is independently selected from the group consisting of hydrogen, C1-C10 alkyl, C1-C10 acyl, C1-C10 hydroxyalkyl, C1-C10 alkoxy, C1-C10 alkoxyalkyl, C1-C10 alkylthio, and C1-C10 alkoxycarbonyl; X and Y are independently selected from the group consisting of —(CH₂)ₘ—, —(CHR⁵)—, —O—, —N—, —NR⁶—, or —S—; Z is —(CHR⁷)—, —C(R⁸)═, or ═C(R⁹)—C(R¹⁰)═; subscript ‘m’ varies from 0 to 4; R⁵ is selected from the group consisting of C1-C10 alkyl, C1-C10 acyl, C1-C10 hydroxyalkyl, and C1-C10 alkoxycarbonyl; R⁶ is selected from the group consisting of hydrogen, C1-C10 alkyl, C1-C10 acyl, C1-C10 hydroxyalkyl, and C1-C10 alkoxycarbonyl; and each of R⁷, R⁸, R⁹ and R¹⁰ are independently selected from the group consisting of hydrogen, C1-C10 alkyl, C1-C10 acyl, C1-C10 hydroxyalkyl, C1-C10 alkoxy, and C1-C10 alkoxycarbonyl.

28. The method of claim 26 wherein the active pharmaceutical agent is pseudoephedrine, and the organoleptic excipient is pyrazines described by Formula I,

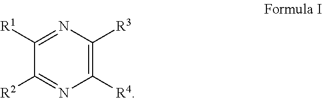

29. The method of claim 28 wherein each of R¹, R², R³, is R⁴ is independently hydrogen, C1-C10 alkyl, C1-C10 acyl, C1-C10 alkoxy.

30. The method of claim 29 wherein R¹ is acetyl, methoxy, or ethoxy; and each of R², R³, and R⁴ is independently hydrogen or C1-C4 alkyl.

31. The method of claim 30 wherein the organoleptic agent is acetylpyrazine, 2-ethoxy-5-methylpyrazine, and 2-ethoxy-6-methylpyrazine.

32. The method of claim 31 wherein said composition is a solid dosage form comprising pseudoephedrine and acetylpyrazine.

33. The method of claim 31 wherein said composition is a liquid dosage form comprising pseudoephedrine and 2-ethoxy-6-methylpyrazine.

34. The method of claim 26 wherein the organoleptic excipient is thiazoles described by structural Formula III,

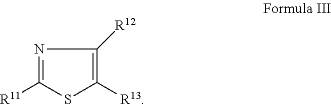

35. The method of claim 34 wherein each of R¹¹, R¹², and R¹³ is independently hydrogen, C1-C10 alkyl, C1-C10 acyl, C1-C10 hydroxyalkyl, C1-C10 alkoxy, C1-C10 alkoxyalkyl, C1-C10 alkylthio, or C1-C10 alkoxycarbonyl.

36. The method of claim 35 wherein each of R¹¹, R¹², and R¹³ is independently hydrogen, C1-C10 alkyl, or C1-C10 acyl.

37. The method of claim 36 wherein each of R¹¹ and R¹² is independently hydrogen or methyl, and R¹³ is acetyl.

38. The method of claim 37 wherein the organoleptic agent is 2-acetylthiazole, 4-acetylthiazole, 5-acetylthiazole, 5-acetyl-2,4-dimethylthiazole, 2-acetyl-4,5-dimethyl-thiazole, and 4-acetyl-2,5-dimethylthiazole.

39. The method of claim 38 wherein said composition is a solid or a liquid dosage form comprising pseudoephedrine and 5-acetyl-2,4-dimethylthiazole.
